(19) United States
(12) Patent Application Publication
(10) Pub. No.: US 2022/0406442 A1
(43) Pub. Date: Dec. 22, 2022

(54) DONOR CRITERIA AND ALERT NOTIFICATION SYSTEMS

(71) Applicant: CERNER INNOVATION, INC., Kansas City, KS (US)

(72) Inventors: Anna-Therese Fowler, Leawood, KS (US); Alex Lende, Kansas City, MO (US); Eric Wilson, Lee's Summit, MO (US); Mayur Rajendran, Overland Park, KS (US); Taylor Floyd, Kansas City, MO (US); Jay Vaglio, Mission, KC (US)

(21) Appl. No.: 17/354,487

(22) Filed: Jun. 22, 2021

(51) Int. Cl. G16H 40/20; G16H 10/60; G06F 16/951

(52) U.S. Cl. CPC G16H 40/20; G16H 10/60; G06F 16/951

(57) ABSTRACT

Systems, methods, and devices in accordance with embodiments automatically provide notifications to clinicians regarding tissue (including organs and fluids) that are eligible or ineligible for donation. In some cases, information about a potential donor is crawled, including from third-party sources, and comprehensive data for a potential donor is automatically analyzed against donor criteria, to provide an alert to remote devices without requiring accessing of full medical records for the potential donor.

### BACKGROUND

Systems and devices facilitate processes for donation of tissue, such as organs, to patients in need. For example, registries exist for patients to sign-up as potential recipients of tissue donated from a deceased individual, and in some cases tissue eligible for donation is tracked. This tracked information does not relate to the patient as a whole, for example to evaluate or identify tissue for donation, this information is not readily- and remotely-accessible by clinicians in near real-time in order to improve and increase donations. No comprehensive system for donation exists, and the “harvest” rate of potentially-eligible tissues for donation is very low, for example as low as 2%. There is a need for computerized systems and methods to meaningfully notify clinicians of a deceased individual and/or tissue available for donation, for example, using information from multiple or disparate sources and/or including tracking or obtaining health information overall for the individual.

### SUMMARY

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter. The present invention is defined by the claims.

In brief and at a high level, this disclosure describes, among other things, methods, systems, and computer-storage media for notifying clinicians regarding tissue for donation in near real-time. Embodiments include computerized systems programmed or configured to receive an indication of expiration for an individual input into a remote computing device, determine the individual is a donor, and receive electronic health information associated with the individual. Systems can also receive one or more exclusionary criteria for donor tissue, identify a first ineligible tissue from the individual based on the electronic health information and the one or more exclusionary criteria, and provide a notification of the first ineligible tissue on the remote computing device. In embodiments, the indication of expiration is an input relating to the individual being terminal, which can trigger the determining, by the system, that the individual is a donor. Embodiments include identifying a first likely eligible tissue from the individual based on the electronic health information and the exclusionary criteria, and receiving medical condition information from a third-party source using Fast Healthcare Interoperability Resources (FHIR). In some cases, the electronic health information associated with the individual uses a different format than the medical condition information. In some cases, systems according to embodiments integrate the electronic health information with the medical condition information for a comparison to the one or more exclusionary criteria for donor tissue.

Embodiments include aspects where electronic health information is received from an electronic health record, and the system integrates the electronic health information with the medical condition information received using FHIR by combining the electronic health information with the medical condition information for simultaneous comparison to the one or more exclusionary criteria for donor tissue. In some cases, lung tissue can be determined to be ineligible, or in some cases eye, kidney, blood, and/or other tissues are determined to be ineligible or eligible (meaning likely eligible in some cases). In embodiments, some systems, such as internal systems or medical records, use a first framework or format, while remote or third-party sources can be accessed using other formats such as FHIR, PowerChart, and/or HealtheIntent.

Embodiments include computerized systems for determining a list of one or more tissues for donation in near real-time, including receiving first medical information about a patient, automatically requesting, by the computerized system based on the first medical information, second medical information about the patient, and receiving one or more rules relating to potential tissue donation by the patient. Embodiments include analyzing the first and second medical information based on the rules, and automatically providing an alert to one or more remote devices that indicates the one or more tissues for donation. In some cases, an alert is a pop-up window. In embodiments, the computerized system is automatically prompted to request the second medical information from one or more third-party sources based on the first medical information. In some cases, the first medical information relates to an expiration of the patient.

Embodiments also include one or more computing devices programmed to perform a method for notifying a clinician of potential tissue to be donated from an individual without the clinician accessing one or more medical records for the individual, the method including crawling one or more data sources for information about the individual, wherein the information corresponds to one or more donor criteria, receiving an indication the individual is terminal or expired, and automatically providing a near real-time notification to one or more clinicians indicating a donor-eligibility status for one or more tissues of the individual. In some cases, the donor-eligibility status of tissues indicates that a first tissue is eligible and a second tissue is ineligible. In embodiments, crawling the data sources for information about the individual includes accessing the data sources using an FHIR format.

### BRIEF DESCRIPTION OF THE DRAWINGS

Embodiments are described in detail below with reference to the attached drawing figures, wherein:

FIG. 1 is a block diagram of an exemplary computing environment suitable to implement embodiments of the present invention;

FIG. 2 is an exemplary user interface component suitable to implement embodiments of the present invention;

FIG. 3 is an exemplary user interface component suitable to implement embodiments of the present invention;

FIG. 4 is a flow chart illustrating one or more steps or actions performed by implementations involving systems according to an embodiment of the present invention;

FIG. 5 is a flow chart illustrating one or more steps or actions performed by implementations involving systems according to an embodiment of the present invention;

FIG. 6 is a flow chart illustrating one or more steps or actions performed by implementations involving systems according to an embodiment of the present invention; and

FIG. 7 is a flow chart illustrating one or more steps or actions performed by implementations involving systems according to an embodiment of the present invention

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

There are needs for improved systems and devices such as embodiments of the present invention. Various facilities or services, such as medical facilities or other entities involved in patient care or treatment or care of deceased individuals, can use systems to attempt to register a patient for a potential transplant, or to attempt to track potential tissue including organs for donation. For example, an individual's identification can identify the individual as a donor, which can be entered into an EMR, but this approach fails to gain the attention of clinicians at an appropriate time to consider donation. Clinicians pronouncing individuals as deceased are required, or relied upon, to identify any tissue such as organs that may be usable for donation to a patient. For example, a deceased individual due to the opioid epidemic may not be recognized, in time, as possessing salvageable organs for donation.

Current systems are not satisfactory and result in a lack of real-time or near real-time information and notifications. Current systems fail to obtain information from various sources, and they fail to consider electronic information from electronic medical records or other sources, for example to determine tissue that is eligible or ineligible for donations prior to or at the time of death. Additionally, current systems fail to provide sufficient or timely notifications relating to potential donations from deceased individuals to patients in need.

As one example, at a time point on Mar. 8, 2019, approximately 113,691 patients were identified as being in need of tissue donation, such as organ transplants, with 74,540 being active waiting-list candidates. On the other hand, approximately 500,000 individuals expired in a care facility that was suitable or optimal for harvesting donations from individuals, but only about 5% were classified as potential donors. Fewer than 40% of potential donor individuals were actually used for tissue donation, with a national average of about three organs donated per individual. This can result in an approximately 2% harvest or donation rate. Improved methods and systems for computerized notifications or alerts relating to potential donors and specific tissue for potential donations can improve the donation rate to patients in need, which can improve and save lives.

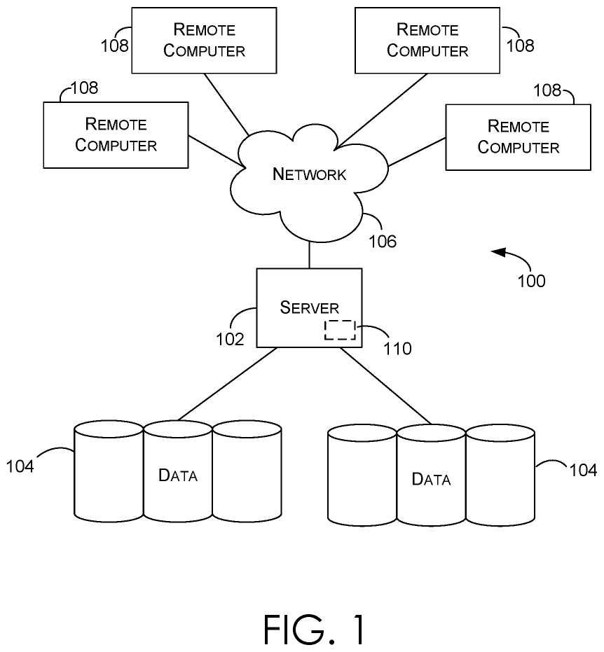

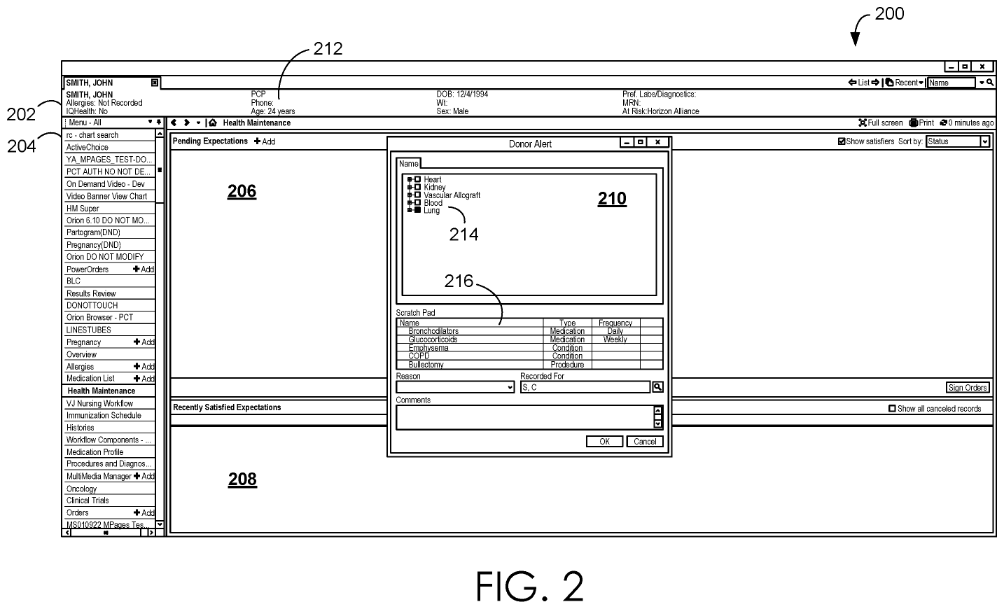

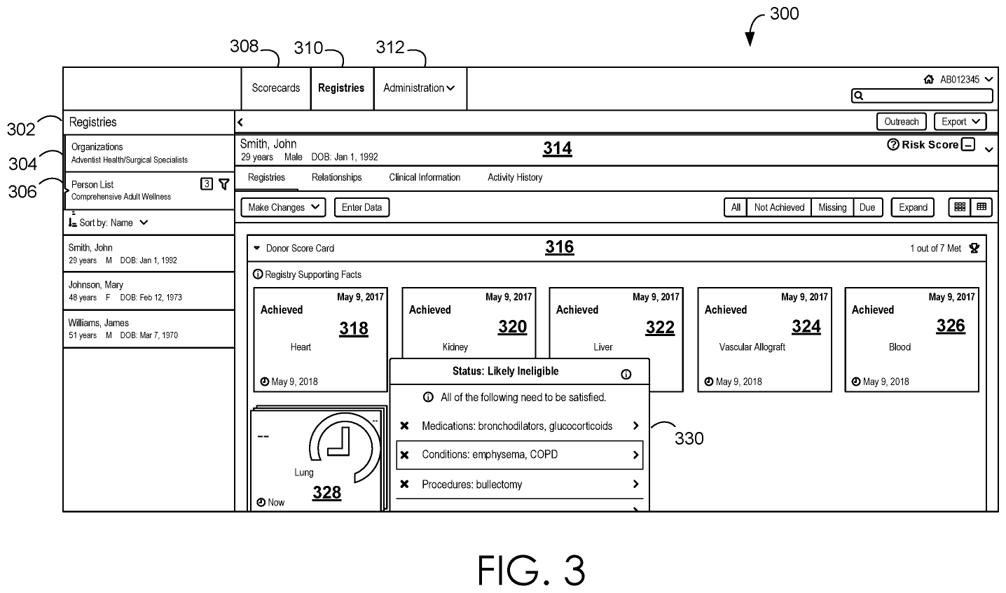

FIG. 1 shows an exemplary diagram 100 for use with one or more embodiments of the present invention. In one example, systems and methods according to embodiments include a server 102 in communication with one or more data stores such as a databases 104. Server 102 is also connected to a network 106, and the network 106 can communicate with one or more remote computing devices 108, as shown in the example in FIG. 1. Remote computing devices 108 can include a user device, for example a tablet used by one or more medical professionals such as a clinician. In some cases, a clinician uses a remote computing device 108 to access or view a user interface (for example one or more user interfaces as discussed with respect to FIGS. 2 and 3, below). In embodiments, databases 104 can include data from one or more electronic medical records (EMRs) and/or other data from internal or third-party sources, as discussed in more detail below. In embodiments, systems and methods can access one or more other sources to obtain patient information or supplemental patient information.

In an exemplary embodiment, a server 102 includes program components 110, which can access or receive data from, and communicate data to, data stores such as databases 104. Program components 110 can also obtain or receive data from remote computing devices 108 and transmit or provide data to remote computing devices 108. Embodiments crawl one or more data sources, in some cases in near real-time, such as databases 104 and/or remote computing devices 108. In some cases, program components 110 crawl data sources for relevant health information regarding a recently-deceased individual or an individual at a time prior or proximate to becoming deceased. Databases 104 include third-party data sources, in embodiments, such as third-party medical facility or provider records, pharmacy records, data relating to medical procedures performed at one or more facilities, and test results or records for an individual such as genetic tests or results, for example. One or more remote computing devices 108 can be (or have access to) third-party data sources, in some cases.

Embodiments of the present invention provide integration with FHIR systems or data, which can enable data from third-party data sources to be used or consumed by program components 110. For example, program components 110 can crawl, or request and/or receive, data in an FHIR format and use it, including for consideration in real-time or near real-time, in accordance with embodiments. Aspects of program components 110 can use one or more components to crawl, ingest, and/or process data from FHIR-based systems or storage. For example, program components 110 can parse data in an FHIR format in order to apply rules or processes to the data (along with other health data, for example from EMRs or a clinician) to determine if certain tissues are found sufficient for donation (“whitelisted”) or not sufficient (“blacklisted”) and/or to provide a notification to a clinician of potential donation opportunities.

Because FHIR-system data can be crawled or used by program components 110, systems and methods in accordance with embodiments can provide cross-vendor support. For example, FHIR data can be accessed using an application programming interface (API) to request or retrieve data from an FHIR-based system. In embodiments, one or more databases 104 includes FHIR-based or -accessible data that can be obtained for an individual using an API request. In some cases, one or more remote computing devices 108 can include FHIR data or be used to execute an API request to obtain FHIR data, which can be received by program components 110. Other data formats besides FHIR can be accessed or used by systems and methods in order to cause program components 110 to receive data from multiple sources, including sources using various data elements or storage formats. For example, program components 110 can request or receive data from two or more different databases 104, parse or isolate certain data values (such as a diagnosis or treatment received, or a demographic or other health data as discussed below), and utilize this data in combination with data from other sources (such as EMRs) to comprehensively evaluate a potential donor individual and notify a clinician of available tissue for donation. Information from sources can be used as or converted into FHIR, for example under the HL7 standards.

Program components 110 can be an application or applet and, in some cases, can be distributed on one or more devices, including one or more remote computing devices 108. In one example, a remote computing device 108 is a tablet used by a clinician to access program components 110 via one or more user interfaces, such as user interfaces 200, 300 discussed in more detail below. In embodiments, program components 110 are running or accessible in the background and can act to provide an alert, such as a pop-up window or notification, during use of one or more other programs by a clinician. For example, a clinician may use a remote computing device 108 to input medical records or other information about an individual receiving medical care or an evaluation. As the clinician interacts with remote computing device 108, certain inputs or other actions can trigger program components 110 to access potential donor information for the individual, or actions can trigger program components 110 to calculate or determine potential donor information in near real-time for the individual. Even if the clinician is using another program, for example relating to bed capacity in a facility or scheduling, a notification (as discussed in more detail below) can be displayed on the remote computing device 108 so that a clinician can be notified of potential donor information associated with the individual while the individual is still in the clinician's care or immediate vicinity or before an amount of time passes that will negatively impact the potential donation.

As one example, a deceased individual due to a drug overdose is a potential donor individual for tissue that is determined as not affected by the cause of death or any other medical treatments or conditions. The individual could be relatively young for a potential donor individual, and although certain tissue is determined to be affected or likely affected (for example due to a diagnosis of liver cancer or damage), other tissue such as eyes or skin may be determined to be unaffected and/or suitable for donation (e.g., “whitelisted”). More techniques and improvements for donation of tissue such as organs, skin, and blood have occurred over time, particularly for tissue such as skin where donations have become more feasible, but many donation opportunities are missed due to lack of notice and/or lack of screening ahead of time and/or in near real-time. Methods and systems described herein can address the low percentage of donor individuals, including those with potential donations that are not recognized, or not recognized in time, by current systems.

Embodiments provide a multifaceted approach to prevent waste on the medical side, for example by considering potential donations near a time when donations may be appropriate or possible, and by enabling and improving the selection of individuals as donors. For example, program components 110 may request or receive information from third-party sources (for example as stored at a database 104) to determine an individual has selected to be a donor. Program components 110 may also receive information from an FHIR system and extract or use certain data, such as donor status, medication history, treatments, etc. In one example, a medication history from a third-party source may indicate a pathology such as a disease associated with a potential donor's eyes, causing that donor's eyes to be eliminated from consideration for donation, even though the potential donor's cause of death and present medical situation were unrelated to any eye disease. On the other hand, program components 110 can retrieve information from one or more data sources, in near real-time as a potential donor receives end-of-life care or other treatment, to determine that no medical history indicates any adverse conditions or treatments associated with the potential donor's eyes, and program components 110 can cause a notification to be received by a clinician on a remote computing device 108 indicating the potential donor's eyes appear satisfactory for a donation or transplant. Embodiments can consider all available data at a point in real time that will benefit a clinician and enable more donations.

As another example, program components 110 can determine, from received data from one or more sources, that data in a potential donor's medical history eliminates one or more tissues from potential donation. For example, a medication, treatment, or diagnosis received by program components 110 (for example as determined from FHIR data from a third party, such as prescription data for a certain chemotherapy drug) can indicate a potential donor individual has or had cancer. This determination can trigger further requests for data or analysis by program components 110, in embodiments, which can be used to determine the type or stage of cancer and which tissue to eliminate for donation based on the data. In this example, program components 110 determine a progressive stage of cancer has reached certain tissues, based on the received data. For example, biopsy or diagnosis records can be analyzed by program components 110 to evaluate certain tissue and to determine that certain tissue, such as a specific organ (e.g., the liver) is not eligible for donation or is too high of a risk for donation. In some cases, program components 110 determine that certain issues impact more than one type of tissue, even if that tissue itself is not flagged or noted as impacted in the data. For example, if a stage 4 cancer is determined to exist in certain tissue, then other tissues from that patient may also be identified as not eligible for donation, due to the risk of spreading cancer. In another example, certain blood diseases could be identified and program components 110 determine one or more blood diseases also impact (and eliminate for donation) other tissues besides blood such as the heart or plasma, due to the presence of a particular disease and its known potential impact on other tissue.

In some cases, embodiments of the invention can proactively analyze individuals as potential donors. For example, methods and systems can include server 102 or another component analyzing data about an individual and identifying one or more gaps in the data records, or one or more potential additional data sources (for example based on an EMR that notes a treatment or visit at a facility without full accompanying data). The information associated with any such gaps can be requested from other sources via network 106. Alternatively, any information about the individual can be requested and/or received from identified third-party sources, such as sources mentioned in an EMR or sources that are flagged as associated or potentially associated with the individual.

In embodiments, server 102 receives data, from internal and/or third-party sources, regarding an individual on a periodic basis, such as once a year. In this example, program components 110 annually automatically request and/or receive data about an individual from one or more sources, including sources that are accessed or communicate in FHIR or other data formats. For example, program components 110 can crawl sources and identify data about the individual, or program components 110 can send API requests to one or more sources regarding the individual. The data could be a donor registration status according to a government database, medication or treatment information such as a surgery or emergency room visit, or other information about the individual. For example, information about health or lifestyle could be pulled or received, such as whether the individual participated in a program to quit smoking, or a study regarding an experimental treatment for a condition. Program components 110 can identify from a third-party source, for example, that an individual was in a rehabilitation facility on certain dates relating to drug use, and the specific drug(s) could be identified. This information can be considered, along with other available health and lifestyle information, to identify tissue as eligible or ineligible for donation.

Embodiments described herein can automatically notify a clinician while the clinician is reviewing health information related to a patient. For example, when an EMR for a patient is accessed, a notification can be generated by a program component 110 indicating an individual qualifies as a potential donor for one or more tissues. In some cases, a notification can specify one or more tissues, such as blood or kidney, for example, for which an individual qualifies as a potential donor, based on the information accessed by the system from one or more sources. In embodiments, when a medical professional enters information into a patient's EMR, this triggers the system to determine the potential donor status of the individual. The system can determine that the individual is currently a donor, in embodiments. In other cases, when triggered by an update to the individual's medical information, the system can generate an alert or notification to be displayed to the medical professional, and in some cases the system determines the individual's donor status is not available, for example because it is unknown or undecided. In some cases, the notification may require acknowledgement and/or closing in order to continue with other actions via an interface, such as entering more information into an EMR.

In embodiments, the notification that a patient is not registered as a donor, according to the system, can be triggered by an input that would typically be made by a medical professional, so that the notification is provided in real time during a consultation with a medical professional. This allows a program component 110 to request an update with respect to an individual's donor status at a time when a medical professional using an interface (with the notification) likely has access to the individual. Such a notification can cause the medical professional to discuss tissue donation with the individual, which may lead to an increase in registered donors. For example, the individual may decide to be listed as a donor, which the medical professional can immediately update, or the individual may inform the medical professional that the individual is a donor, which can be updated in the system. The medical professional can encourage potential donors to consider registering and answer questions. Because the system can provide a near real-time notification, which can be specific to donor registration and require acknowledgement, the system is likely to obtain additional donor information over time, to improve future instances involving the individual's as donors. For instance, they system can generate notifications relating to missing donor status information in the system for one or more patients. The system can receive updates from medical professionals regarding donor status, for example after consultations, and the system can use this updated information to notify other medical professionals of potential donor tissue from the patients in the future.

In some cases, the system can determine an individual's likelihood of registering as a donor. This likelihood can be used by the system to determine whether a notification will be presented to a medical professional regarding donor registration, in embodiments. For example, if an individual has a likelihood of registering as a donor that is above a threshold, such as a 50% likelihood or a 65% likelihood, and it is determined the individual is not currently a donor, then the system may decide to send a notification to a medical professional regarding that individual. By only sending a notification regarding a potential donor when the individual has a higher likelihood of registering, the system can increase donor registrations while avoiding wasting medical resources such as medical professional's consultation time. In embodiments, an alert or notification may be presented for any individuals not registered as a donor, but the notification will only require acknowledgement, such as closing out the notification to continue, if the individual is determined to be likely to become a registered donor.

The system can consider one or more factors to determine an individual's likelihood of registering as a donor at a particular time. In some cases, the factor considered may be whether the individual is participating in a routine or check-up type of appointment. For example, if an individual has a medical visit for an annual physical, or a scheduled ultrasound, or a work-clearance type of appointment, and the individual is not determined to be a registered donor, the system may display a notification that requests donor information and/or reminds a medical professional to discuss donor registration with the individual. In other cases, the system may compare the available information about the patient to other patients, in order to determine if similar patients (for example of the same age and/or with similar health conditions) are likely to register as donors. In embodiments, other demographic data may be used for this determination, such as geographic location, education level, etc., which can be used to cluster individuals into groups, determine the likelihood of donor registration for one or more groups, and determine which group corresponds to a current patient, for purposes of obtaining a likelihood for the current patient. In an exemplary embodiment, the patient's history can be analyzed for any prior conversations regarding tissue donation, which can be used to determine a patient is likely or not become a donor, or which can be weighed with the other factors discussed above to determine a likelihood. The likelihood can cause notification(s) to discuss donor registration. In some cases, the likelihood can cause a notification to a medical provider and/or to a patient after a visit, such as through a patient portal, to register as a donor or to update one or more records relating to donation. In embodiments, a notification sent after a patient visit can be identified by the system and used to cause a second notification during the next patient visit regarding organ donation.

In other embodiments, systems in accordance with the present invention can request and/or receive data about an individual, for purposes of considering a potential donation from the individual, on a monthly basis or another regular time frame, for example by crawling sources of data and storing information about the individual (e.g., in database 104). In some cases, the proactive or regular analyses of individuals can be updated in near real-time when an individual checks into a medical facility, or when an individual is considered terminal, or when an individual is recently deceased. In other cases, the analysis of an individual for purposes of donating tissue according to embodiments only occurs in near real-time when an individual is deceased or near being deceased.

In some cases, embodiments allow a notification to be provided to a clinician regarding a deceased individual prior to removal or transport of the individual's body to another location, or prior to decisions that may render tissue less desirable or unusable for donation, such as prior to removal of certain machines such as a ventilator or other life support services, or prior to circulation or other functions ceasing in an individual. One approach can include analyzing all terminal individuals so that program components 110 obtain the most up-to-date information about each individual for rapid consideration and notifications upon an individual expiring. During this process, one or more tissues per individual can be determined to be appropriate for donation, and/or one or more tissues per individual can be determined to be inappropriate for donation. This determination can be subject to a last-minute real-time check by program components 110 upon expiration of an individual and prior to a notification to a clinician on a remote computing device 108 that displays which tissues are eligible for donation. Embodiments include program components 110 implementing positive feedback loops to capture information and/or to continue to capture information, so that the analysis upon expiration of an individual is less sudden and based on more data and more up-to-date data, including data from disparate sources. In some cases tissue can be classified as suitable or unsuitable, or flagged for consideration, for example at an approximate time of death, which can mean prior to transport of a deceased individual and/or prior to expiration of an opportunity to donate tissue due to passage of time, temperature or circulation issues, etc.

FIG. 2 illustrates an exemplary user interface 200 in accordance with embodiments of the present invention. User interface 200 is one example of a screen view, for example on a remote computing device 108, from the perspective of a clinician. A clinician may access an EMR or other information about an individual, in this case “John Smith,” by selecting or navigating to tab 202. For instance, tab 202 can be selected by a clinician when the individual John Smith is considered terminal or recently deceased. A menu 204 as shown in the example in FIG. 2 can indicate potential selections by a clinician, for example with options to add information such as medications. Menu 204, in some cases, indicates sources or types of information about the individual John Smith. A clinician also has an option to input or add pending expectations 206, as shown in the upper frame or window of FIG. 2, and to view or otherwise manipulate recently satisfied expectations 208. User interface 200 includes an example of a donor alert 210, which in some cases overlays or pops-up over the other content being displayed.

FIG. 2 includes an example of data about the individual (John Smith), as shown at individual data 212, for example his age. This information can be used along with other received information from internal or EMR data and/or from third-party sources, for example data in an FHIR format, to make determinations that cause program components 110 to trigger the display of a donor alert 210. The donor alert 210 can open or expand onto a user interface 200 based on opening an individual's EMR (which can trigger a determination as to eligible tissue for donation), or based on entering information into an individual's EMR or other records, such as entry of an individual into hospice or a diagnosis of the individual as terminal (which could also trigger a determination as to eligible tissue for donation). In some cases, the entry of accessing of health information for an individual, such as inputting that a procedure was not successful, can automatically cause program components 110 to gather information in near real-time regarding the individual's history, demographics, and other behaviors or diagnoses, which can cause program components 110 to automatically generate a donor alert 210. The donor alert 210 is automatically triggered by computerized systems in accordance with embodiments herein, such that a clinician is alerted to potential tissue donations prior to next step(s) regarding an individual, in order to facilitate a successful donation.

Donor alert 210 includes tissue indication 214 in some cases, which can indicate to a clinician the specific tissue(s) that are eligible or ineligible, or recommended or not recommended, for donation. In the example in FIG. 2, tissue indication 214 shows multiple tissues “whitelisted” or eligible for donation, including “Heart,” “Kidney,” “Vascular Allograft,” and “Blood.” On the other hand, the tissue “Lung” is indicated as not eligible or recommended, for example due to the filled-in icon next to “Lung,” as shown in FIG. 2. Each of the tissue types displayed on donor alert 210 could be selectable for more information, and in some cases the recommendation can be changed or overridden.

The exemplary donor alert 210 in FIG. 2 includes notes 216, which can display more information about an individual, such as medications taken and their frequency levels. In some cases, a clinician can add information to notes 216 regarding the individual. Notes 216 can expand to display more information in response to selection of one or more tissues from tissue indication 214. In embodiments, aspects of the notes 216 can be hovered over or selected to display additional information, such as the start date of a medication.

The illustrative user interface 200 in FIG. 2 relates to individual John Smith, in this example a 24-year-old male. Embodiments of the present invention analyze data about John Smith, for example in near real-time to John Smith's time of death, from multiple sources. The sources could have been crawled beforehand and/or could be queried for data or new data upon an entry into a record that John Smith is deceased. Program components 110 can query or receive John Smith's EMR data, which can include current chart data for John Smith, along with information regarding John Smith's medication usage over time and other information, such as whether John Smith was a smoker and, if so, for how long and at during what time periods.

Program components 110 can also retrieve or access rules regarding tissue donation, such as state or national rules or rules developed by organizations focused on tissue donation or particular tissue types, such as a heart or lung organization, or a blood donation organization, or another entity with rule-setting or oversight activities relating to tissue donation. The rules or recommended practices can be accessed by program components 110 from remote sources, or stored in a data store such as database 104. In embodiments, program components 110 can be programmed with certain predetermined rules for tissue donation, and in some cases updates could be pushed to program components 110 or requested on a periodic basis or in response to notices. For example, the guidelines or protocols for lung donation can be part of a library or data store of information that program components 110 can use or access to determine eligible tissue for donation.

In the example shown in FIG. 2, John Smith's “Lung” tissue has been disqualified by program components 110, based on data about John Smith's health, healthcare, demographics, and/or activities. Also as shown in FIG. 2, John Smith's other tissues as shown in tissue indication 214 are not disqualified and can be used or considered for donation, based on the analysis of John Smith's information. Program components 110 determined, prior to triggering the donor alert 210, that John Smith should be considered for potential donations and retrieved known health information about John Smith from one or more databases 104, along with requesting unknown information about John Smith from one or more third-parties, and integrating this information for use by program components 110. For example, John Smith may have been designated as terminally ill, and at that point (and/or at each point when John Smith's information is accessed after such designation), program components 110 can evaluate all available information regarding John Smith for purposes of notifying a clinician of eligible (and ineligible) tissues for donation in time for such donations to occur, for example in near real-time before treatment of the individual changes or the individual is moved. Embodiments compare information about John Smith to the rules for donation that are stored or accessed to determine if each tissue meets the criteria for donation. As shown, John Smith's “Heart” and “Kidney” tissues, for example, were determined by program components 110 to be eligible for donation, or to not be off limits or ineligible, based on a comparison of the rules to the data (including data extracted or received by program components 110 in FHIR format and converted or extracted for use by program components 110 along with EMR information.

For example, one or more rules or protocols regarding tissue donation from third-party sources can provide a checklist for comparison to data about an individual. An organization associated with lung health or lung donations can issue rules that program components 110 use as a checklist, for example being a non-smoker, being under a certain age, lacking exposure to asbestos, etc. Each item on a checklist can be compared to John Smith's data as collected by program components 110. In this example, John Smith has not met the checklist requirements for eligible “Lung” tissue donation, but he has met checklist requirements for “Heart” and “Kidney” tissue donation, as shown. Because the system can crawl data and compare data to checklist requirements in advance, systems in accordance with embodiments can provide near real-time information on tissue, such as ineligible tissue for donation, to aid the clinician and improve donation rates. The analysis can be dynamic and can continue to compare supplemental data or new information to rules, which can also be updated over time. Embodiments raise clinicians awareness of potential donor individuals at the time and in the setting where an individual is most likely to be able to donate tissue. The example shown in FIG. 2 includes notes 216, which in this case show two medications (one taken daily and one taken weekly), two conditions, and one procedure.

In some cases, computerized systems in accordance with embodiments can access or display donor information, such as a donor alert 210, without bringing up or opening an entire chart or EMR for the individual. This can provide a timely notification that otherwise may not be delivered until it is too late for the individual to donate. In some cases the notes 216 display partial medical information that is relevant to the rules for particular tissue donations for consideration or review, without accessing full or other medical information. In one example, program components 110 can identify missing information regarding an individual that is relevant to one or more donor checklist requirements or donor scorecards, request or obtain this information to the extent possible, compare the individual's information to the requirements automatically, and store this analysis for fast access at a time of potential donor consideration, for example in database 104. Continuing with this example, based on information about the individual being accessed or viewed by a clinician, or entered by a clinician (such as a terminal illness or a status as deceased), systems using program components 110 can then access this analysis (and in some cases update it) in near real-time, so that the system or a clinician does not need to access full medical information or request the analysis regarding potential donations in order to receive a notification such as donor alert 210.

In some instances, certain rules for donors are set by facilities or administrators and used by embodiments of the present invention. For example, program components 110 can access stored or configured rules such as a donor scorecard or checklist from a database 104 or from a remote source via network 104, or certain rules can be programmed and accessed by server 102. A facility may select to configure computerized systems disclosed herein to override other rules or criteria, for example from an organization or government. If a certain medical facility does not have available personnel or equipment to facilitate donations or preservation of tissue, the computerized system can be configured to suppress certain donor alerts 210 that would otherwise be issued, or such configuration could be based on issues relating to liability or suspensions of donations for other reasons. In other examples, if a high need exists or an experimental donation practice is being clinically tested, program components 110 can be configured to identify eligible tissues for donation that may not otherwise qualify based on an analysis of available information and published or national donor criteria.

Embodiments provide a framework for interoperability with any other system to enable timely and accurate patient information in the context of various EMRs and other electronic sources. For example, this framework can analyze information from Millennium® software and/or Epic-brand software, EHRs and/or EMRs, and other third-party data sources for example using FHIR. Program components 110 can ingest data from such sources, for example as received from one or more remote computing devices 108, and consider this information in near real-time to provide a donor alert 210 proximate to the time of death and/or more likely to be prior to transfer or further treatment of an individual. For example, basis information needed by program components 110 to compare an individual's data with one or more donor criteria can be transmitted (for example in response to API requests) in an FHIR context, thus information from third-party sources can be requested and received by program components 110 in an FHIR framework or format that can be used to process a donor alert 210 for display.

In accordance with embodiments, existing internal data sources of an entity relating to patient health can be culled in near real-time, along with data retrieved and/or stored in an external format such as FHIR, which can also be culled in near real-time. At or near the time of an individual's death, embodiments can compare comprehensive patient data from the various data sources to certain rules or checklist donor criteria to eliminate or rule eligible various tissues, including organs and/or fluids in some cases, for donation. In some cases, the best available information or a preliminary set of data is compared to donor criteria in advance of an individual becoming deceased, to save some or all computational time closer to a time of death, depending on whether additional data is available to be culled at that time. In embodiments, program components 110 can monitor or receive flags or status information from one or more remote sources, for example as embodied on one or more remote computing devices 108, when additional data is available or may be available to be considered prior to determining whether one or more tissues are eligible or not for donation.

As shown in FIG. 2, for example, a recently-expired individual such as John Smith has a medication history of bronchodilators and glucocorticoids, a condition list of emphysema and COPD, and a procedure list of bullectomy. The output of program components 110 relating to John Smith can indicate, after analysis, that John Smith's lung tissue is disqualified for donation while other tissues, including organs and fluids such as blood, are likely qualified. In embodiments, this information that is relevant to an analysis by program components 110 regarding donation of tissue is displayed on user interface 200 without requiring a clinician or system to open or access all information about John Smith and without requiring any navigation to review data regarding John Smith with respect to tissue donation. Instead, in embodiments, the analysis by program components 110 is performed automatically based on certain health information regarding John Smith or his admittance into a certain portion of a facility such as hospice or intensive care.

Turning now to FIG. 3, an exemplary user interface 300 is provided in accordance with embodiments. As shown in the left-side panel, tabs can be available for selection or display such as a registries tab 302, an organizations tab 304, and a person list tab 306. For example, this user interface 300 can be used to view potential donors or the status of individuals as potential donors currently or in the future. Selectable tabs or options such as scorecards option 308, registries option 310, and administration option 312 exist, which can show or allow input of criteria for donors from various sources such as donor scorecards or registries, or by administrators. Here John Smith is displayed from a person list 306, and an individual screen portion 314 for John Smith is thus displayed. A donor score card 316 for John Smith is also displayed, in embodiments as a portion of or overlaying an individual screen portion 314.

As shown in FIG. 3, multiple potential tissues associated with John Smith have been considered by program components 110 for potential donation status as eligible or ineligible. In this example, the heart indicator 318 displays “Achieved” which can mean heart tissue from John Smith is eligible for donation and/or meets one or more checklists of criteria for donation of heart tissue, and for example John Smith's heart tissue could be “whitelisted” for near real-time providing of notifications to clinicians, as long as no further or contradicting information is detected, in embodiments. The kidney indicator 320, liver indicator 322, vascular allograft indicator 324, and blood indicator 326 also indicate an “Achieved” status, which can cause these tissues to be marked eligible on an alert or “whitelisted” for faster analysis in the future in near real-time. On the other hand, the lung indicator 328 in this example indicates that John Smith's lung tissue is not achieved, for example because an achieved status is still pending. Additionally, status indicator 330 indicates that John Smith's lung tissue is “Likely Ineligible” or potentially ruled ineligible, in some cases. Part of the status indicator 330 can display one or more conditions or other failed criteria that support the status (such as “Conditions emphysema, COPD”) in some cases, which can be selected for further information.

In embodiments user interfaces 200 and 300 are asynchronous including options to update or view tabs in a portion of an interface while other data remains displayed on a second portion, for example an individual screen portion 314 could remain fixed while selections are made among tabs such as registries tab 302, organizations tab 304, and/or person list tab 306, or among other aspects of user interface 300. As another example, individual screen portion 314 could remain displayed (in this example for an individual John Smith) while a donor score card 316 is viewed or required, or while a notification 318 is displayed, for example pop-up or selectable window as shown at notification 318 in FIG. 3.

As shown in the exemplary user interfaces 200, 300 in FIGS. 2 and 3, embodiments can provide solutions in the form of medical charts or be accessible from a view of medical charts, or in a dashboard view, for example to allow navigation to various information about a patient such as John Smith, or other patients such as Mary Johnson. In some cases, methods and systems described herein can be integrated into a PowerChart and/or HealtheIntent framework, including data received using FHIP or other formats to obtain data from sources, with a minimum of one touch point as an option. In a PowerChart framework, for example, immediate feedback can be provided to a clinician, such as a donor alert 210, and in a HealtheIntent framework, for example, information can be aggregated and/or used to provide actionable content, which can also be in the form of a donor alert 210.

The term tissue as used herein in the context of tissue donations can also include organs and/or fluids, such as commonly donated tissues including heart, lung, kidneys, liver, and blood, and/or other tissues that may be relatively less-commonly donated in some cases, such as face, eyes, ovaries, and limbs. In some cases, certain exclusion criteria for tissue donations may depend on the tissue itself and/or on a donation center or regulatory entity, for example the Centers for Disease Control has mandated certain criteria, such as all individuals with potential tissue donations (or the tissue itself) are to be screened for HIV and hepatitis C, in order to prevent the spread of these communicable diseases during or because of a transplant process.

Table 1 shows exemplary standard donor criteria for certain tissue donations, which can be based, for example, on published or accepted standards, such as checklists of requirements issued by organizations of medical professionals and/or advisors relating to donations. Table 1 includes exemplary criteria for heart, lung, and kidney tissue, which can be obtained by program components 110 and/or programmed or configured into program components 110. The standard donor criteria can be expanded in certain circumstances based on patient need or other circumstances such as severe shortages, shorter-term donations, and/or recipient life expectancy.

TABLE 1
Exemplary Donor Criteria for Certain Tissue Donations
Heart
Age < 55
No history of chest trauma or cardiac disease
No prolonged hypotension or hypoxemia
Appropriate hemodynamics
Mean arterial pressure > 60 mmHg
Central venous pressure 8 to 12 mmHg
Inotropic support less than 10 mg/kg/min (dopamine or dobutamine)
Normal electrocardiogram
Normal echocardiogram
Normal cardiac angiography (if indicated by donor age and history)
Negative serology (hepatitis B surface antigen, hepatitis C virus and human
immunodeficiency virus)
Lung
Age 20-45
PaO2:FiO2 > 350
Smoking history None
Chest X-ray Clear
Ventilation days < 5
Microbiology Gram stain negative
Bronchoscopy Clear
Ischemic time < 4 hours
Kidney
Age < 60
Cause of death Cerebrovascular accident (+/−)
No history of hypertension
Terminal Serum Creatinine > 1.5 mg/dl

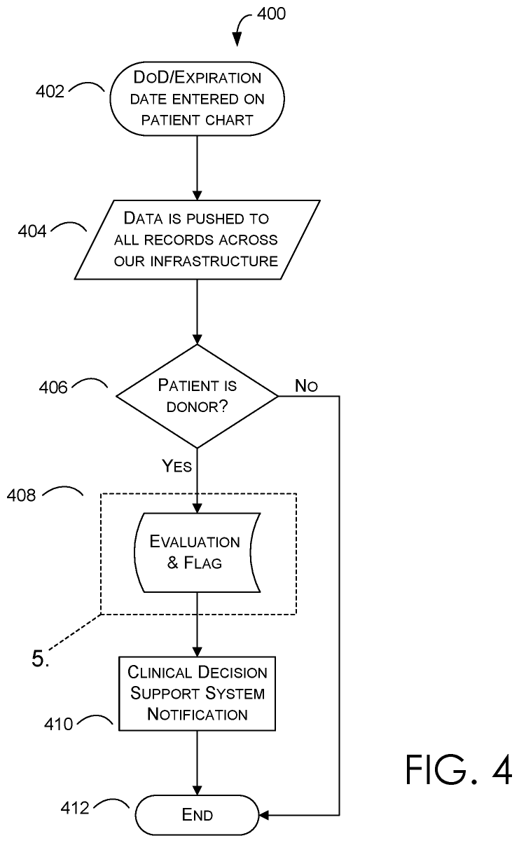

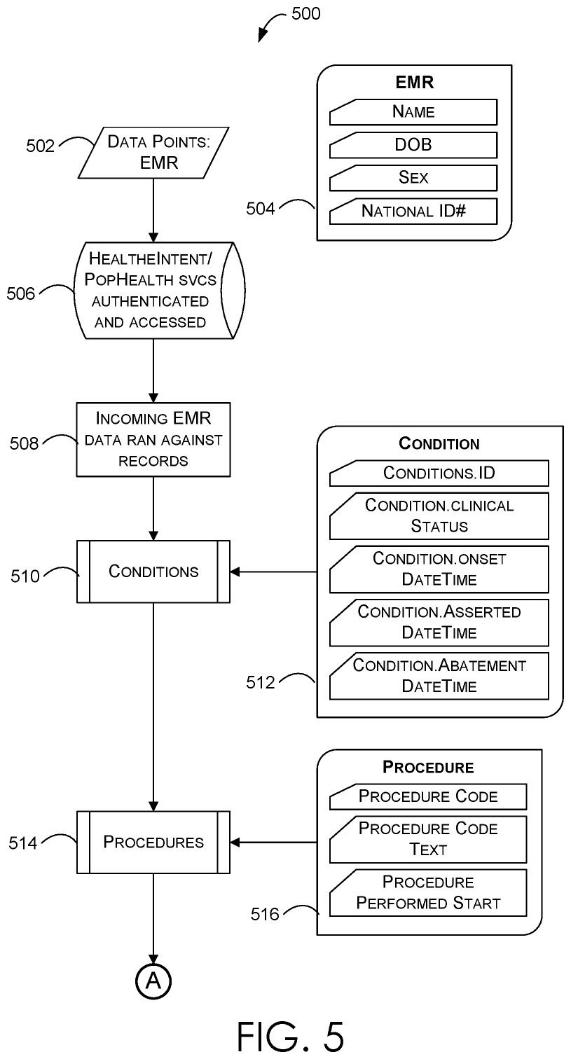

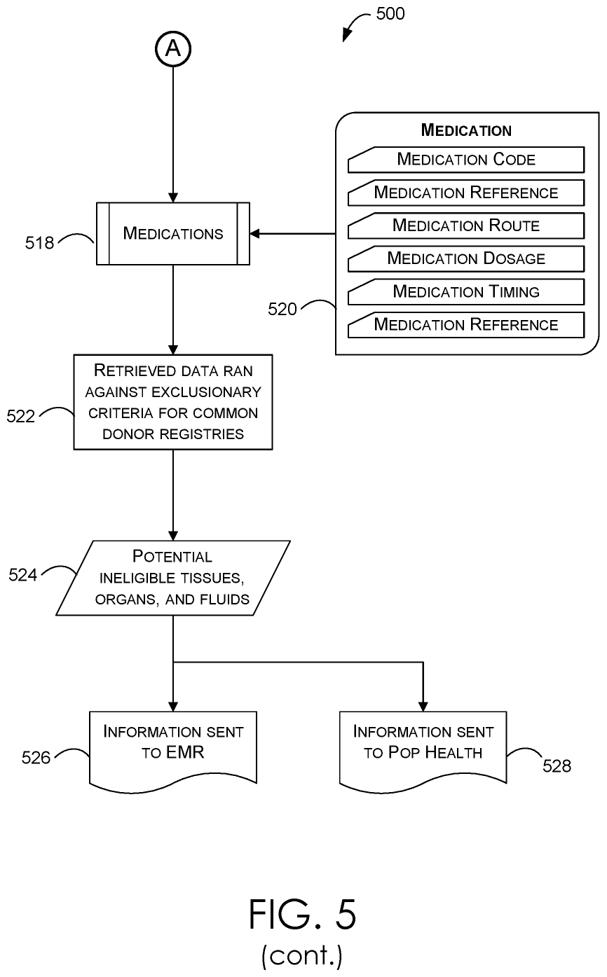

Turning now to FIG. 4, a flow diagram 400 illustrates one or more steps in accordance with embodiments of the present invention. At step 402, a date of death (“DoD”) or expiration date is entered into an individual patient's chart, such as an electronic record. The entry of this information can trigger the computerized system to automatically proceed to the next step. At step 404, sub-processes are automatically performed due to the date entered into the system at step 402, which can begin with data being pushed to all records across infrastructure (regarding bed turnover, staff notifications, etc., by a system). The triggered process continues at step 406 by determining whether the expired individual is a donor. If the individual is a donor (for example according to stored information that is or was obtained from a government entity in a real-time analysis), the process proceeds automatically to step 408 for an evaluation and/or flagging step, shown in more detail in FIG. 5. After the evaluation and/or flagging step 408, the process continues automatically to step 410, clinical decision support system notification, such as a donor alert 210 shown in FIG. 2. The flagging process can cause an individual to be maintained or not moved as providers or clinicians are notified by the computerized system, for example as shown in FIG. 5 and as discussed herein. The process proceeds to end at step 412. If the individual is not determined to be a donor at step 406, the process also proceeds to end at step 412.

Turning now to FIG. 5, one or more aspects of the evaluation and/or flagging process, for example as shown at step 408 in FIG. 4, are illustrated. FIG. 5 shows a flow diagram 500 in accordance with embodiments, for example as triggered and automatically performed by a computerized system based on the input indicating a deceased individual, such as step 402 in FIG. 4, or automatically performed based on both such an input and an automatic determination that the individual is a donor, as shown at step 406 in FIG. 4. At step 502, one or more data points can be obtained or received, such as EMR information 504 as shown in FIG. 5. At step 506, other sources such as systems using HealtheIntent and/or PopHealthCare services, or other sources, can be queried, or data from such sources can be sent to and received by, for example, program components 110, in some cases using an FHIR framework for requesting and/or receiving data. At step 508, incoming EMR data is run against records, which can be done to fill-in gaps in the available data about an individual or to identify gaps and cause additional queries (not shown). In embodiments, the received medical record data for an individual is combined with other sources that are authenticated and accessed (e.g., at step 506) or used to supplement it, such that all available data from internal and/or remote sources are used and considered by the computerized system. At step 510, conditions are received or considered, with, for example, one or more of the conditions aspects 512 shown in FIG. 5, such as the onset date and time or abatement date and time.

At step 514, procedures are received or considered, for example the procedure data 516 for each procedure received at step 514 in FIG. 5. Procedures could be medical procedures, such as treatments or surgeries, or a diagnosis, and the procedure data 516 can include one or more procedure codes, textual information, and/or timing information related to the procedure. Continuing with FIG. 5, at step 518, medication information 520 relating to one or more medications taken by an individual is received. For example, program components 110 in FIG. 1 can be an aspect of server 102 and receive or request medication information 520, which can include dosage, method of ingestion or treatment, one or more codes, and other timing or medication information 520. At step 522, some or all retrieved data is run against exclusionary criteria, such as donor checklist requirements or other rules, such as rules from common donor registries or other national or accepted standards, or configurations set by administrators.

At step 524 potential ineligible tissues, including organs and/or fluids, are automatically identified by the computerized system. For example, “whitelisted” or other eligible tissues, such as heart, lung, blood, skin, etc., can be identified by the system, in some cases because program components 110 automatically analyze the prior received information and compare this information to rules or conditions in near real-time. At step 526, information is sent to an EMR (such as flagging a deceased individual as a donor for one or more tissues, in this case including organs or fluids such as eyes and plasma), and at step 528, information is sent to a population health service, such as a PopHealthCare service. In embodiments, information sent to an EMR can reflect or be used to implement a deceased individual acting as a donor or being scheduled for harvest of certain tissues, in some cases in certain orders to preserve tissue (for example, harvesting fluids last, etc.). In some cases, systems such as PopHealthCare can receive information regarding potential tissues for donation, such as skin, organs, or fluids, and use this information to quickly communicate regarding potential donations in a computerized system.

In step 510, when condition aspects 512 are received, for example in response to an API request of third-party health data, the condition aspects 512 can indicate whether a condition is or was chronic or acute and/or the onset time, so that program components 110 can determine the impact of one or more conditions, for example by weighting against thresholds or other rules the amount of time for a condition (e.g., weeks versus decades of a condition) and whether each condition was chronic or acute, which can all be applied comprehensively against criteria that can use this information to make determinations about the likely healthiness, predicted success, and/or eligibility or appropriateness of tissue for donation.

For example, a condition that first occurred fifty years ago and has been chronic or present since its onset is much more likely to meet a criteria for ruling a tissue such as an organ or fluid ineligible. In another example of applying exclusionary donor criteria, for example by program components 110, is the consideration by the system of medication information 520 at step 518, which may ultimately at step 522 rule out one or more tissues for a patient, based on the length of time and amount, for example, of each medication as compared by the system to toxicity information for each medication, in some cases toxicity information specific to certain tissues, such as liver or skin damage, for example, associated with an exposure to a certain amount of medication at once or over time.

In embodiments, the computerized system, for example program components 110, perform one or more steps as described above in order to funnel or filter a list of eligible or potentially eligible tissues to be displayed in a pop-up notification to a clinician via a mobile computing device 108. In step 528, information sent to a “PopHealth” service such as PopHealthCare, or another service for communication among clinicians, is a notification to one or more recipients using a network such as network 106 that one or more tissues are eligible for donation from an individual. The notification of potential donor tissue sent via a “PopHealth” service can provide a location and other information about the individual or available tissue to further facilitate more donations or more efficient donations.

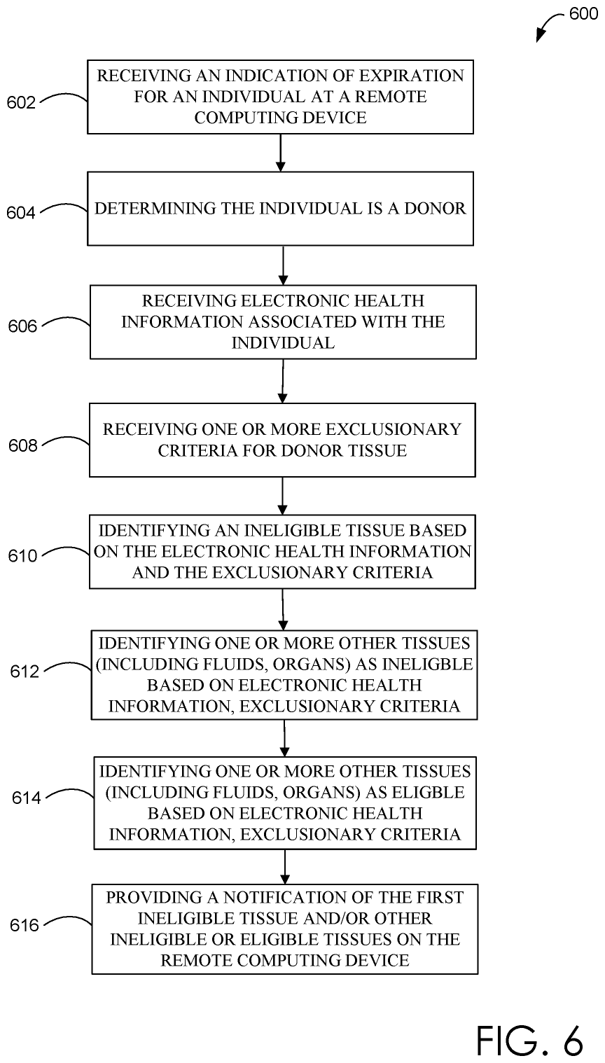

FIG. 6 is a flow diagram 600 showing an illustrative process embodying one or more aspects of the present invention. For example, at step 602, a donor component receives an indication of expiration for an individual via a remote computing device 108. At step 604, the donor component can determine the individual is a donor, for example by accessing medical records and/or third-party data sources. At step 606, the donor component receives electronic health information for the individual, for example from EMRs/EHRs, and in some cases using an internal format or framework that differs from the FHIR framework. At step 608, the donor component receives one or more exclusionary criteria for donor tissue. At step 610, the donor component identifies ineligible tissue based on the health information and criteria, for example the program components 110 can include the donor component and/or a notification component. At step 612, in this exemplary process, program components 110 identify one or more other tissues (including fluids or organs) as ineligible based on electronic health information and one or more exclusionary criteria. Continuing with step 614, one or more other tissues (including fluids or organs) are identified as eligible based on electronic health information and one or more exclusionary criteria. At step 616, the notification component provides a notification of the first ineligible tissue at a remote computing device, for example to notify a clinician in near real-time regarding the individual's potential donations. In one example, a notification can display information indicating the ineligible tissue and/or other ineligible or eligible tissues on the remote computing device, for example blood or lung tissue. In embodiments, part or all of the notification component is embodied on a remote computing device 108, such as a handheld or other portable device.

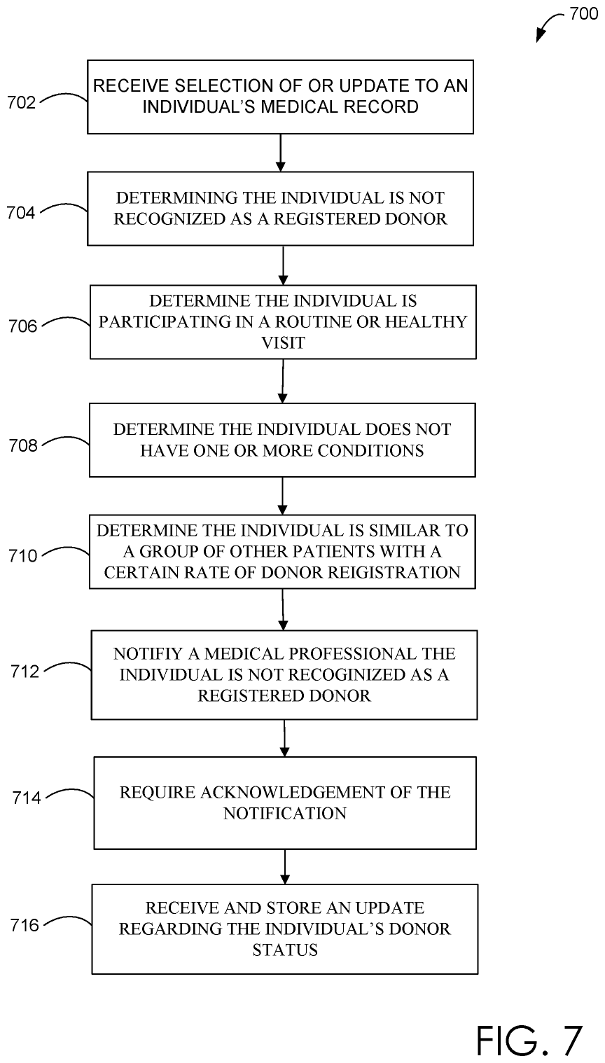

Turning to FIG. 7, an exemplary flow diagram 700 shows aspects of an embodiment. At 702, a selection of a medical record or an update to a medical record is received, for example when a medical professional accesses an EMR or enters information, such as information to indicate a patient visit is occurring. At 704, the system determines the individual is not recognized as a donor, for example according to the EMR and/or according to third-party sources accessed by the system, such as state records regarding donors, for example. At 706, it is determined the individual is participating in a routine or healthy visit, and at 708, it is determined the individual does not have one or more conditions, such as conditions that may disqualify an individual from donating one more tissues, for example a blood or skin disease or condition, or a status as a smoker for a certain number of years, which can be determined from sources such as donor or medical organizations. At 710, it is determined whether the individual is similar to a group of other patients with a certain rate of donor registration. The aspects in 706, 708, and 710 can be used separately or in any combination to determine a likelihood that an individual will become a donor, in order to determine whether to proceed to 712, notifying a medical professional the individual is not recognized as a registered donor. For example, this notification may only be worth implementing in cases where it is determined more likely to lead to a new donor.

At 714, the system can require acknowledgement of the notification to proceed, or in some cases the system will allow the notification to sleep or be minimized then pop up or require acknowledgement prior to closing out of an EMR. At 716, the system can receive and/or store an update regarding the individual's donor status. For example, the system can receive an update that the patient is now a donor, or the system can receive an update that the patient is considering becoming a donor, or is not interested in becoming a donor. During a subsequent visit, the system can access and use this updated information to determine whether to display another notification regarding donor registration. For example, if the patient previously did not register but indicated they would consider becoming a donor, the system can flag this information during the next visit in a notification to a medical provider, for further discussion or updating.

The exemplary modules in diagram 100 and discussed herein are suitable for use in implementing embodiments of the present invention. The modules are merely an example of one suitable computing system environment and are not intended to suggest any limitation as to the scope of use or functionality of embodiments of the present invention. Neither should the computing system environment be interpreted as having any dependency or requirement related to any single module/component or combination of modules/components illustrated therein. Embodiments of the present invention might be operational with numerous other computing system environments or configurations. Examples of computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention might be described in the general context of computer-executable instructions, such as program modules, being executed by a computer. Exemplary program modules comprise routines, programs, objects, components, and data structures that perform particular tasks or implement particular abstract data types. The present invention might be practiced in distributed computing environments where tasks are performed by remote processing devices that are linked through a communications network. In a distributed computing environment, program modules might be located in association with local and/or remote computer storage media (e.g., memory storage devices).

The present invention has been described in relation to particular embodiments, which are intended in all respects to be illustrative rather than restrictive. Further, the present invention is not limited to these embodiments, but variations and modifications may be made without departing from the scope of the present invention.

What is claimed is:

1. A system comprising:
receiving an indication of expiration for an individual input into a remote computing device;
determining the individual is a donor;
receiving electronic health information associated with the individual;
receiving one or more exclusionary criteria for donor tissue;
identifying a first ineligible tissue from the individual based on the electronic health information and the one or more exclusionary criteria; and
providing a notification of the first ineligible tissue on the remote computing device.

2. The system of claim 1, wherein the indication of expiration is an input relating to the individual being terminal.

3. The system of claim 1, wherein the indication of expiration triggers determining, by the system, the individual is a donor.

4. The system of claim 1, further comprising identifying a first likely eligible tissue from the individual based on the electronic health information and the one or more exclusionary criteria.

5. The system of claim 1, further comprising receiving medical condition information from a third-party source using FHIR.

6. The system of claim 5, wherein the electronic health information associated with the individual uses a different format than the medical condition information.

7. The system of claim 6, wherein the system integrates the electronic health information with the medical condition information for a comparison to the one or more exclusionary criteria for donor tissue.

8. The system of claim 7, wherein the electronic health information is received from an electronic health record, and wherein the system integrates the electronic health information with the medical condition information received using FHIR by combining the electronic health information with the medical condition information for simultaneous comparison to the one or more exclusionary criteria for donor tissue.

9. The system of claim 1, further comprising:
identifying a second tissue from the individual as eligible or ineligible based on the electronic health information and the one or more exclusionary criteria; and
providing a notification regarding the second tissue on the remote computing device.

10. A computerized system for determining a list of one or more tissues for donation in near real-time, comprising:
receiving first medical information about a patient;
automatically requesting, by the computerized system based on the first medical information, second medical information about the patient;
receiving one or more rules relating to potential tissue donation by the patient;
analyzing the first and second medical information based on the one or more rules; and
automatically providing an alert to one or more remote devices that indicates the one or more tissues for donation.

11. The system of claim 10, wherein the alert is a pop-up window.

12. The system of claim 10, wherein the second medical information is medical condition information about the patient.

13. The system of claim 12, wherein the medical condition information includes condition onset data.

14. The system of claim 10, wherein the computerized system is automatically prompted to request the second medical information from one or more third-party sources based on the first medical information.

15. The system of claim 14, wherein the first medical information relates to an expiration of the patient.

16. One or more computing devices programmed to perform a method for notifying a clinician of potential tissue to be donated from an individual without the clinician accessing one or more medical records for the individual, the method comprising:
crawling one or more data sources for information about the individual, wherein the information corresponds to one or more donor criteria;
receiving an indication the individual is terminal or expired; and
automatically providing a near real-time notification to one or more clinicians indicating a donor-eligibility status for one or more tissues of the individual.

17. The one or more computing devices of claim 16, wherein the near real-time notification displays on a user interface the donor-eligibility status of one or more tissues of the individual.

18. The one or more computing devices of claim 17, wherein the donor-eligibility status of one or more tissues indicates that a first tissue is eligible and a second tissue is ineligible.

19. The one or more computing devices of claim 16, wherein crawling the one or more data sources for information about the individual includes accessing the one or more data sources using an FHIR format.

20. The one or more computing devices of claim 19, wherein the one or more data sources include data regarding the individual's medication and medical condition history.
